(19) United States
(12) Patent Application Publication
(10) Pub. No.: US 2026/0000853 A1
(43) Pub. Date: Jan. 1, 2026

(54) DISCHARGE HEAD AND LIQUID DISPENSER FOR NASAL APPLICATION OF LIQUID, AND METHOD FOR PRODUCING A DISCHARGE HEAD

(71) Applicant: Aptar Radolfzell GmbH, Radolfzell (DE)

(72) Inventor: Jürgen GREINER-PERTH, Gottmadingen (DE)

(21) Appl. No.: 19/247,012

(22) Filed: Jun. 24, 2025

(30) Foreign Application Priority Data
Jun. 27, 2024 (EP) 24185139.3

(51) Int. Cl. A61M 15/08; A61M 11/00; A61M 15/00

(52) U.S. Cl. CPC A61M 15/08; A61M 11/006; A61M 15/0026; A61M 2207/00

(57) ABSTRACT

A discharge head having a base unit with a coupling device by which the discharge head can be fastened to a pressure accumulator. The discharge head has an actuating unit with an elongate nasal applicator, extending along an applicator axis, and an actuating surface mounted on the base unit to be pivotable about a pivot axis by a hinge device, so that the actuating unit, for the purpose of opening an outlet valve of the pressure accumulator, can be pressed down in a pivoting actuating direction. Components of the discharge head are produced by injection moulds that are separable along one axis.

### CROSS-REFERENCE TO RELATED APPLICATION

This claims priority from European Application No. 24185139.3, filed Jun. 27, 2024, the disclosure of which is hereby incorporated by reference in its entirety.

### FIELD OF APPLICATION AND PRIOR ART

The invention relates to a discharge head for nasal application of liquids from a pressure accumulator. The invention also relates to a liquid dispenser having such a discharge head and to a method for producing such a discharge head.

Discharge heads of the type in question are provided for mounting on a pressure accumulator. They have a base unit for connection, in most cases latching connection, to the pressure accumulator. The pressure accumulator is usually designed as a plastic or metal container with an internal volume of 500 ml or less and has an integrated outlet valve, which has force applied to it from the outside by means of the attached discharge head and can thus be opened. In the pressure accumulator, the liquid that is to be discharged is stored at pressure, usually at an overpressure of at least 1 bar.

For the purpose of valve opening, discharge heads of the type in question have an actuating unit which has a nasal applicator and an actuating surface. When the actuating unit is pressed down, the actuating force is transmitted to the outlet valve and opens the latter. For as long as the actuating unit is pressed down, discharge takes place from a discharge opening on the nasal applicator.

A discharge head according to the invention is used to apply liquid into the nose of the user, in particular in the form of a spray mist of fine droplets. In particular, discharge heads of the type in question are used to discharge saline solution into the nostrils in order to alleviate breathing difficulties.

Discharge heads of the type in question have been disclosed in the prior art, for example in document EP 3412325 A1. The disadvantage of known designs such as the one mentioned above does not lie in the way they work, but primarily in the rather complicated production. The injection moulding process is made more difficult by the fact that, for reasons of comfort, the nasal applicator is usually inclined with respect to a central axis of the base unit. Comparatively complex moulds have been used hitherto, which moulds have to be provided with cores or slides on account of undercuts on the individual components of the discharge head.

### Problem and Solution

The problem addressed by the invention is to make available a discharge head of the type in question, one which is advantageous in terms of its producibility compared to known designs.

To solve this problem, a discharge head for nasal application of liquid from a pressure accumulator is proposed which has a base unit and an actuating unit. These two sub-units are formed as separate sub-units which are connected to each other in the manner described below.

The base unit has a coupling device for coupling to the pressure accumulator. In particular, this can be a coupling device for snap-fit connection to the pressure accumulator. The coupling device can be formed by a circumferential snap-fit collar or by a plurality of snap-fit hooks on the base unit. Other concepts of coupling devices are also conceivable in principle, such as the use of a threaded connection in particular. The base unit is preferably formed by a single component, which is produced by means of plastic injection moulding.

The actuating unit has a nasal applicator and an actuating surface. It is formed by at least one component, particularly preferably by two components, namely an external component and an internal component. The nasal applicator is provided to be inserted into the nostrils of the user. For this purpose, it preferably has an overall elongate shape which, from the actuating surface to a discharge opening at the distal end of the applicator, is longer than the average diameter of the nasal applicator. The nasal applicator defines an applicator axis along its central axis. If, due to the shape of the nasal applicator, no clear central axis can be established, the demoulding direction in the injection moulding production defines the applicator axis within the meaning of this invention.

The applicator axis is preferably inclined with respect to a central axis of the base unit and thus to a central axis of the pressure accumulator, wherein, based on an unactuated state, the angle between the central axis and the applicator axis is preferably at least 10°, particularly preferably at least 15°. However, the angle is preferably not more than 35°. This inclined orientation of the nasal applicator permits comfortable handling when using the liquid dispenser.

The actuating surface is provided fixed in position with respect to the nasal applicator and is in particular arranged offset thereto next to the proximal end of the nasal applicator. The actuating surface is moved with respect to the base unit in the course of the actuation, the nasal applicator being moved together therewith. The actuating surface is preferably provided with elevations in order to allow a secure grip and safe handling.

In the case of a multi-part actuating unit, the nasal applicator and the actuating surface are preferably provided on a common one-piece component.

According to the invention, it is provided that the actuating unit is mounted on the base unit in such a way as to be pivotable about a pivot axis by means of a hinge device, so that the actuating unit, for the purpose of opening a valve of the pressure accumulator, can be pressed down in a pivoting actuating direction with respect to the base unit.

Therefore, in the case of a discharge head according to the invention, there is no integral connection between the base unit and the actuating unit. Instead, said hinge device is formed by a component of the base unit and a component of the actuating unit.

This construction entails that the discharge head is formed of at least two parts which have been produced separately, in particular in each case by means of a plastic injection moulding process. This makes it possible to optimize these two components separately with regard to the injection moulding process, as will be explained below. In addition, the hinge device can also enable a simple form of assembly. Thus, in the course of assembly, the hinge device can first of all be joined, and then the two components connected therewith can be moved relative to each other in a guided manner to their target position.

It is preferably provided that the pivot axis and the actuating surface are arranged on opposite sides of the actuating unit with respect to the nasal applicator. The pivot axis is provided particularly preferably near the nasal applicator, on the side of the actuating unit facing away from the actuating surface. On account of the pivotability, this has the effect, among other things, that a comparatively large actuation distance on the actuating surface leads to a by contrast lesser movement of the nasal applicator. This is advantageous since the user does not pull the nasal applicator back out of the nostril through the actuation movement.

Preferably, the base unit has a base component, which comprises at least the coupling device and a base-unit-side hinge part device. Particularly preferably, this base component can be the sole component of the base unit and can thus form the latter in its entirety.

The base component preferably has a component geometry which is free of non-forcibly demouldable undercuts with respect to a separating direction which corresponds to the central axis of the base unit. This means that the base component can be produced by means of a mould which comprises only injection moulding segments that are movable against each other along a uniform closing and separating direction. Such moulds are much easier to produce than moulds in which slides are provided for the production of undercuts, which slides are to be moved along a direction of movement deviating from a main closing direction/main separating direction during demoulding. Corresponding moulds with a uniform closing and separating direction are not only more cost-effective, they also require less maintenance during operation. They also allow even shorter cycle times.

The base component does not have to be completely free of undercuts for the purpose of easy demoulding. On the contrary, due to the coupling device on the base unit side, it is difficult to completely avoid undercuts. However, according to this further development, it is provided that the only undercuts present on the base component are forcibly demouldable undercuts. Forcibly demouldable undercuts denote regions of the component which are in the way of demoulding, but which nevertheless allow single-axis demoulding through non-destructive deformation of the component. The demoulding therefore takes place with simultaneous elastic yielding of the material of the base component in the region of the forcibly demouldable undercuts.

Like the base component, it is also of particular advantage in the case of the actuating unit, and in particular in the case of an external component of the actuating unit, if this component has a component geometry which is free of non-forcibly demouldable undercuts with respect to a separating direction corresponding to that of the applicator axis.

If both components, i.e. the base component of the base unit and the external component of the actuating unit, are designed in this way, these two main components can be produced very cost-effectively and at short cycle times.

The base unit, in particular said forcibly demouldable base component, has the base-unit-side hinge part device. The latter can preferably be designed in such a way that it has two outer bearing shell portions and, below these outer bearing shell portions and, with respect to a direction transverse to the applicator axis, between the outer bearing shell portions, an opposite inner bearing shell portion. It is therefore proposed that at least three bearing shell portions are provided which, being offset, alternately comprise a region for an axle portion from above and below. Due to their offset arrangement transverse to the central axis and thus transverse to the preferred separating direction of the injection mould, these bearing shell portions can be produced free of undercuts, by two injection moulding segments each defining the respective shape of each of the bearing portions from above and below.

In order to achieve this advantageous design, it is preferably provided that, for the purpose of demoulding of the base component in injection moulding production with respect to the central axis, no portions of the base component are present above the inner bearing shell portion and below the outer bearing shell portions. The bearing shell portions therefore form upper and lower end surfaces of the base component in relation to the separating direction.

As regards the actuating-unit-side hinge part device, it is preferably provided, for the purpose of easy production, that the hinge part device has a central axle carrier, which has a bearing contact surface on its underside, and has, on both sides of this axle carrier, two axle portions which lie opposite each other and which have a bearing contact surface on their top.

The hinge part device on the actuating unit side is thus formed to match the aforementioned construction of the base-side hinge part device. The underside of the axle carrier forms the axle region that is in contact with the central bearing shell portion of the base part. The two axle portions, which are provided on both sides of the axle carrier and protrude freely from the latter, form, with their tops, the bearing contact surfaces which at least in sections abut the two outer bearing shell portions.

For the purpose of easy demoulding of the external component on which the actuating-unit-side hinge part device is provided, it is preferably provided that, with respect to the applicator axis, no portions of the external component are present above the top of the axle portions below the underside of the axle carrier. The tops and undersides of the axle carrier and of the axle portions thus form upper and lower end surfaces of the external component in relation to the separating direction.

On their side facing the axis, the bearing shell portions of a discharge head according to the invention preferably have, at least in some regions, a circular cylindrical or rotationally symmetrical surface which, together with the circular cylindrical shape of the axle portions and the underside of the axle carrier, allow pivoting to take place with minimal friction and minimal jolts.

The described design of the bearing shell portions, of the axle carrier and of the axle portions therefore represents a possibility of being able to produce the external component of the actuating unit and/or the base component in the injection moulding process without slides.

Moreover, the two aforementioned components are preferably designed in such a way that production can be carried out with a single-axis injection mould, that is to say with a cavity-forming mould which is composed of at least two injection moulding segments, of which the injection moulding segments which delimit the cavity are spaced apart from each other only in one separating direction for demoulding.

In particular, on a side facing away from the hinge device, the actuating unit, in particular the external component of the actuating unit, can have a skirt extending in the direction of the applicator axis. Due to its extent being in the direction of the applicator axis, the demoulding of this skirt does not pose a problem.

It is further preferable that the actuating unit has at least one outwardly projecting stop wing, which limits the mobility of the actuating unit counter to the actuating direction by striking against a corresponding holding surface of the base unit. Preferably, at least two stop wings are provided which lie opposite each other on both sides of the actuating unit. The one or more stop wings preferably have a stop surface whose normal vector does not match the applicator axis. Nevertheless, the stop wings do not prevent the use of a single-axis injection mould if, for the purpose of ensuring single-axis demouldability of the external component with respect to the applicator axis, no portions of the external component are present above and below that of the stop wing.

During the use of the liquid dispenser, the at least one stop wing serves the purpose of defining an end position to which the actuating unit can pivot back after a discharge has taken place. In addition, the stop wings can serve the purpose of taking up mounting forces during assembly, as will be explained below.

As has already been described above, a discharge head according to the invention has a hinge device which permits the pivotability of base unit and actuating unit. In principle, the assignment of the two hinge part devices to the base unit and the actuating unit, as described above, is not mandatory.

In general terms, the following construction is proposed in which it is not defined which of the hinge part devices is provided on which unit. According to this construction, the hinge device comprises a first hinge part device and a second hinge part device.

The first hinge part device forms a hinge bearing and has at least two outer bearing shell portions and, arranged opposite them in the direction of the applicator axis, an inner bearing shell portion. The second hinge part device forms a hinge axle and has a central axle carrier, which is mounted on the inner bearing shell portion, and two freely projecting axle portions which are aligned therewith and which are mounted on the outer bearing shell portions.

Regardless of whether this or another construction of the hinge device is used, it is preferred that a joining direction of the hinge device, in the direction of which the hinge part devices of the actuating unit and of the base unit can be coupled, is angled relative to a central axis of the base unit, preferably by an angle of at least 60°, particularly preferably by an angle of at least 80°, in particular by approximately 90°.

The central axis of the base unit defines a direction that corresponds to the direction of mounting of the discharge head on the pressure accumulator. If the hinge device, as proposed above, has a joining direction that deviates from the mounting direction, this is useful for assembly, since there is less risk of the hinge part devices separating from each other when the discharge head is pressed onto the pressure accumulator. This preferred joining direction of the hinge part device and the above-mentioned stop wings together ensure that, when mounting the discharge head on a pressure accumulator, the actuating unit takes up a defined position relative to the base unit and can also absorb forces in the joining direction.

It is particularly preferably provided, with regard to the hinge device, that the bearing shell portions extend inward from an inner side of the base component substantially orthogonally with respect to the central axis of the base component.

The hinge device is preferably designed with some play. This not only helps the actuating unit to move smoothly relative to the base unit, but can also enable a desired relative movement in translation. For this purpose, the hinge part devices are preferably formed in such a way that, in a coupled state, they are movable to a limited extent with respect to each other in the joining direction in a manner free of deformation, preferably by a displacement path of at least 0.2 mm, in particular by at least 0.4 mm.

This relative mobility results in improved interaction between the actuating unit and the valve on the pressure accumulator side. The pivotability of the actuating unit proposed here has the effect that the distance between the valve and a sub-portion of the actuating unit acting on the latter, in particular a connector nozzle on the actuating unit which is coupled to a valve body of the outlet valve, is able to change transversely with respect to the central axis of the base unit, which in principle entails the risk of jamming. The displaceability, which the hinge device according to this further development allows, can compensate for this and can have the effect that the distance between the valve and the sub-portion of the actuating unit acting thereon is variable to a lesser extent, if at all, in the transverse direction. This enables operation free of disturbance.

Preferably, a discharge head according to the invention has an actuation protection, which comprises a protection portion which is integrally formed on the actuating unit or the base unit. The protection portion has at least one destructible sub-portion, which can be destroyed by an actuating displacement of the actuating unit with respect to the base unit.

Such a protection portion can serve several purposes. It can prevent unwanted actuation of the liquid dispenser, for example in luggage, and it can indicate that a first use has not yet taken place. In the present case, the protection portion is preferably an integral part of the external component or of the base component. It can have one or more destructible sub-portions that are destroyed when the liquid dispenser is put into service, for example in the form of plastic bridges that are cut through on initial actuation.

Preferably, the protection portion is designed such that, after destruction of the at least one destructible sub-portion, no complete separation of a part of the protection portion from the base component or from one of the components of the actuating unit occurs. Particularly preferably, a stable extension on the inner side of one of the components forms an inwardly projecting carrier, between which and the inner wall of the base component destructible plastic bridges are provided. Upon first actuation, the bridge or bridges are destroyed, but the carrier with the separated bridges remains on the base component.

A discharge head of the type according to the invention has at least two components, without taking into account the protective cap that can be attached to the base unit. Preferably, however, the actuating unit is at least in two parts, so that, in addition to an external component, an internal component is also provided.

The external component comprises at least the actuating surface, which forms the actuating-unit-side hinge part device and a sleeve portion of the nasal applicator including a discharge opening. The internal component is permanently connected to the outer part, for example by a clamping connection. It forms a line portion by means of which liquid can be directed from the pressure accumulator to the discharge opening. In particular, the internal component can form a connector nozzle for transmitting force to the outlet valve of the pressure accumulator and/or an insert which extends into the nasal applicator.

The use of an internal component separate from the external component simplifies production, since the external component can be produced, in particular by this measure, in a single-axis separable injection mould.

As was explained at the outset, the discharge head described is used as part of a liquid dispenser that comprises a pressure accumulator with integrated outlet valve. The actuating unit pushes onto a valve body of this outlet valve when manual actuation takes place. The outlet valve opens and the liquid can flow from the pressure accumulator into the discharge head as far as the discharge opening. The liquid pressure and/or geometries adapted for this purpose in front of the discharge opening can enable the generation of a spray jet.

The liquid dispenser is preferably filled with a pharmaceutical liquid. For the purposes of the invention, this also includes saline solutions which can be dispensed for the relief of breathing difficulties.

As has already been explained in the context of the description of the discharge head, the advantages of the described design lie in the simplicity of production of the individual parts and of handling during assembly.

The invention also includes new methods for producing the individual parts and for assembly.

As regards the production of a base component, which comprises a base-unit-side hinge part device having at least one lower bearing shell portion and at least one upper bearing shell portion, it is proposed that the production is carried out by means of an injection moulding process, in which a plurality of injection moulding segments are used, which can be displaced along a common closing and separating direction. In the course of production by this method, the injection moulding segments are brought together to form a cavity corresponding to the base component, plastic material is injected into the cavity, and, after curing, the injection moulding segments are displaced with respect to each other in the common opening and separating direction for the purpose of demoulding (single-axis injection mould separation).

The comparatively simple form of the base component and the design of the hinge part device permit the use of such a single-axis injection mould.

For the production of the hinge part device, it is advantageous if, for the purpose of demoulding, at least one first injection moulding segment, which is adjacent to a top of the at least one upper bearing shell portion and to an upper inner side of the at least one lower bearing shell portion of the base component, is spaced apart from at least one second injection moulding segment, which is adjacent to an underside of the at least one lower bearing shell portion and a lower inner side of the at least one upper bearing shell portion.

As regards the production of an external component, which has an actuating-unit-side hinge part device, wherein this hinge part device has a central axle carrier which has a bearing contact surface on its underside, and wherein the hinge part device has, on both sides of the axle carrier, two axle portions which lie opposite each other and which have a bearing contact surface on their top, it is likewise proposed that the production is carried out by means of an injection moulding process in which a plurality of injection moulding segments are used, which can be displaced along a common closing and separating direction.

Here too, in the course of production, the injection moulding segments are brought together to form a cavity corresponding to the external component, plastic material is injected into the cavity, and, after curing, the injection moulding segments are displaced with respect to each other in the common closing and separating direction for the purpose of demoulding (single-axis injection mould separation). For the purpose of demoulding, preferably at least one first injection moulding segment, which is adjacent to a top of the axle portions, is spaced apart from at least one second injection moulding segment, which is adjacent to an underside of the central axle carrier.

Furthermore, an advantageous assembly method is proposed. This assembly method is used to mount a discharge head with a base unit and an actuating unit onto a pressure accumulator. In this case, the actuating unit has at least one outwardly projecting stop wing of the kind described, which limits the mobility of the actuating unit counter to the actuating direction by striking against a corresponding holding surface of the base unit. Furthermore, the actuating unit has a connector nozzle for transmitting force to an outlet valve of the pressure accumulator. This connector nozzle is preferably penetrated by the outlet channel.

During the mounting of the discharge head onto the pressure accumulator, a snap-fit connection is established on the one hand between the coupling device of the base unit and a locking edge of the pressure accumulator. On the other hand, the connector nozzle is coupled to the outlet valve of the pressure accumulator, for example by pushing it into a valve body of the outlet valve.

The mounting force required for this is applied via the base unit, in particular via a protective cap attached to the base unit.

As the discharge head is being pressed onto the pressure accumulator, the at least one stop wing serves to transmit force from the base unit to the actuating unit, so that the connector nozzle of the actuating unit can be coupled to the outlet valve by means of this force. The one or more stop wings are designed in such a way that the force to be expected in this case can be transmitted without destroying the one or more stop wings.

Particularly preferably, it is provided that the hinge device has been joined before the discharge head is snap-fitted onto the pressure accumulator, wherein preferably hereby the stop wings reach the region of the holding surfaces on the base unit. The mounting force can then be transferred from the hinge device and the stop wings together from the base unit to the actuating unit and thus to the connector nozzle.

### BRIEF DESCRIPTION OF THE DRAWINGS

Further advantages and aspects of the invention will emerge from the claims and from the following description of preferred exemplary embodiments of the invention, which are explained below on the basis of the figures.

FIGS. 1A and 1B show a liquid dispenser according to the invention, respectively in section and not in section.

FIG. 2 shows a discharge head of the liquid dispenser in a separate view.

FIG. 3 shows the individual parts of the discharge head in an exploded view.

FIGS. 4A to 4C show components of the discharge head from further perspectives.

FIG. 5 illustrates the details of the hinge device of the discharge head.

FIGS. 6A to 8 illustrate the injection moulding process for two of the components of the discharge head, and also the assembly process.

### DETAILED DESCRIPTION OF THE EXEMPLARY EMBODIMENTS

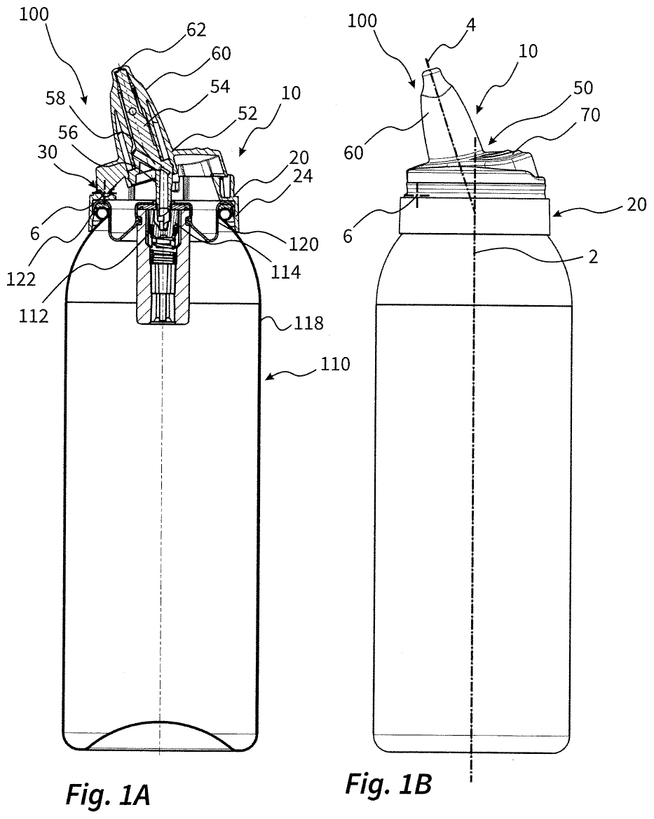

FIGS. 1A and 1B show an overall view of a liquid dispenser 100 according to the invention, respectively in section and not in section.

According to the present exemplary embodiments, a liquid dispenser 100 according to the invention has a pressure accumulator 110, the outer surfaces of which are formed by a metallic accumulator body 118 and a partially metallic lid 120. It could instead have a plastic accumulator body or lid.

The discharge head 10 is latched onto a locking edge 122 of the lid 120. For this purpose, it has a base unit 20, which is formed by a one-piece base component 22. This base component 22 has an annular outer wall, on the inside of which the coupling device 24 is provided in the form of a circumferential or interrupted locking web, which is snapped onto the locking edge 122.

An actuating unit 50 of the discharge head 10 is pivotably hinged on the base component 22. For this purpose, the base component 22 and an external component 52 of the actuating unit 50 are connected to each other by means of a hinge device consisting of two hinge part devices 30, 90. The hinge device defines the pivot axis 6 of the actuating unit 50.

By means of its external component 52, the actuating unit 50 forms the outer shell of a nasal applicator 60 which extends along an applicator axis 4 and at the distal end of which a discharge opening 62 is provided. Furthermore, by means of an internal component 54, which is latched onto an inner contour of the nasal applicator 60, the actuating unit 50 forms a liquid channel 58 which extends from a connector nozzle 56 into the nasal applicator 60.

The connector nozzle 56 extends through a central opening of the lid 120 into a valve body 114 of the outlet valve 112.

The liquid dispenser 100 is actuated by the user first inserting the nasal applicator 60 into a nostril and then manually pressing down the actuating surface 70. When this is done, the entire actuating unit 50 pivots clockwise by approximately 5° in relation to FIGS. 1A and 1B. The connector nozzle 56 presses on the valve body 114 and distances it from a sealing surface of the outlet valve, so that the pressurized liquid flows from the pressure accumulator 110 into the discharge head and up to the discharge opening 62 and is there discharged in the form of a spray jet.

The design of the discharge head 10 is optimized in particular with regard to a simple and cost-effective production process. The discharge head 10 consists of said three components, that is to say the base component 22 of the base unit 20 and also the external component 52 and the internal component 54 of the actuating unit 50. In addition, the discharge head can have a protective cap as the fourth component of the discharge head 10.

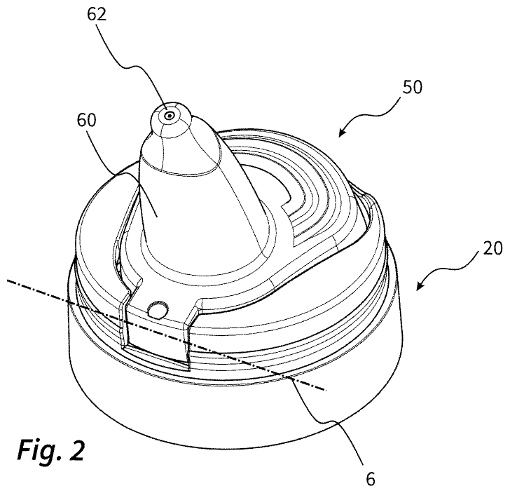

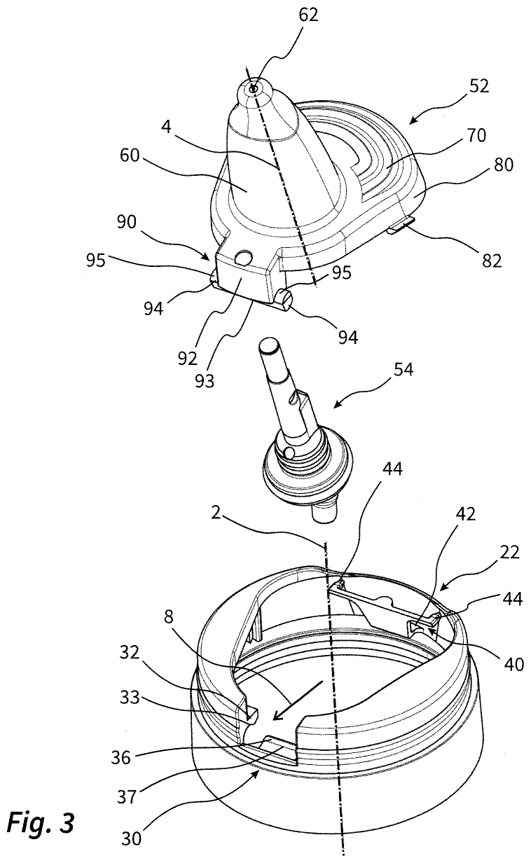

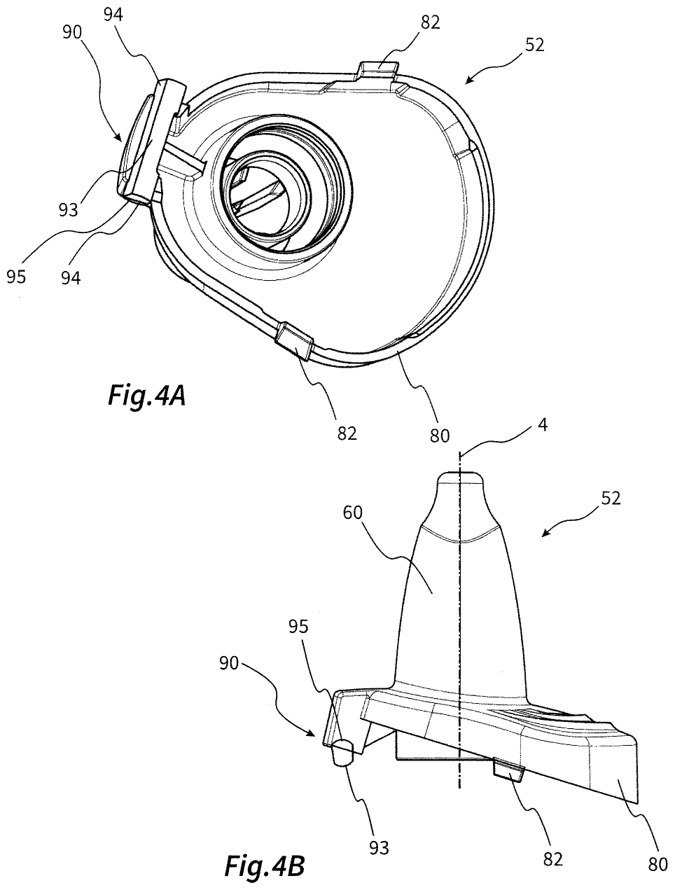

FIG. 2 shows the discharge head 10 again, in a separate view. FIG. 3 shows the three components 22, 52, 54 of the discharge head 10 in an exploded view. FIGS. 4A and 4B show the external component 52 in further perspectives. FIG. 4C shows the base component in a separate view. The details of the discharge head 10 are explained below using these figures.

The hinge device formed from the two hinge part devices 30, 90 on the external component 52 and the base component 22 is designed in such a way that it enables particularly simple production of the components as injection-moulded components. The hinge part device 30 provided on the base component 22 has three bearing shell portions 32, 36. The bearing shell portions 32 form outer and downwardly directed bearing shells 33. The central bearing shell portion 36 forms a deeper bearing shell portion with an upwardly directed bearing shell 37. Correspondingly, the hinge part device 90 on the external component 52 has a central axle carrier 92, the underside of which forms a curved bearing contact surface 93, which is provided for reception in the bearing shell portion 36. On both sides of the axle carrier 92, freely projecting axle portions 94 are provided which have domed bearing contact surfaces 95 on their top.

In FIG. 3, the joining direction is illustrated by the arrow 8, in which direction the hinge part device 90 is inserted into the hinge part device 30. The joining direction encloses an angle of about 90° with the central axis 2 of the base component 22.

In order to limit the pivotability of the actuating unit 50 with respect to the base unit 20, stop wings 82 are provided on both sides of a skirt 80 of the external component 52. These stop wings 82 are dimensioned so that the external component 52, with the internal component 54 already inserted, can be inserted through the free opening on the top of the base component 22, before it is moved in the direction of arrow 8 for the purpose of joining the hinge device 30, 90 together. In such an assembled state, the stop wings 82 are located below holding surfaces 38 of the base component 22. As can be seen in FIG. 4B, the stop wings 82 are inclined with respect to the applicator axis 4, wherein they protrude freely to the side, and no further portions of the external component 52 are present above and below the stop wings 82.

In addition to the function of the stop wings 82 to limit the back-pivoting of the actuating unit 50 with respect to the base unit 20 during operation, they fulfil a function during assembly, which is explained in the following.

As a protection against tampering, the discharge head 10 has an actuation protection 40. The latter is provided in the form of a protection portion 42 which faces inwards from the annular outer wall of the base component 22 and which has a T-shape. From this protection portion 42, and in a manner offset from the central attachment of the protection portion to the annular outer wall and above this attachment, two destructible bridge portions 44 likewise extend to the annular outer wall. These bridge portions 44 are positioned in such a way that the skirt 80 cuts through these bridge portions 44 when the actuating unit 50 is pressed down for the first time, whereupon, on account of the central attachment of the protection portion not being affected by this, no part detaches from the base component 22.

The actuation protection 40 primarily serves in this case to prevent unintentional actuation of the liquid dispenser, for example in luggage. It can also serve the purpose of visually indicating whether the liquid dispenser has already been put into operation and whether an initial discharge has taken place. This can be achieved, for example, if a gap between the skirt 80 and the annular wall of the base component 22 is sufficiently large that the bridge portions 44 are visible through this gap before initial use.

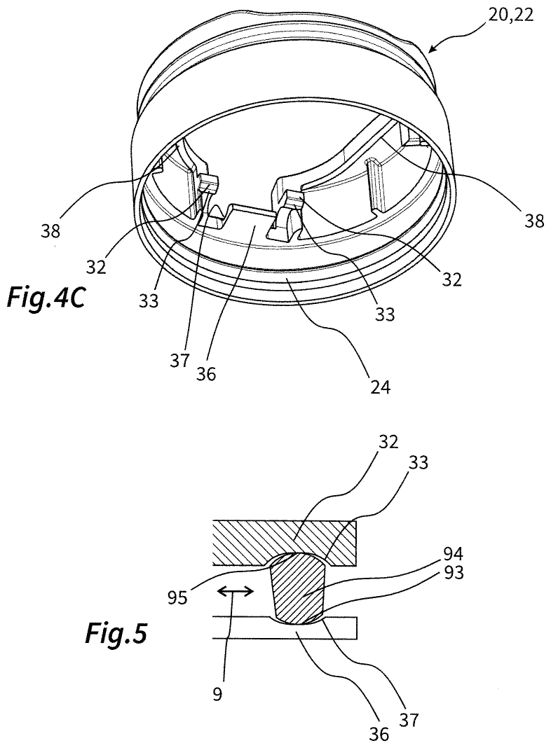

FIG. 5 illustrates a special function of the hinge device 30, 90. The design of the bearing shells 33, 37 is chosen such that the axle carrier 92 and the axle portions 94 and thus the actuating unit 50 maintain a mobility in the direction of the arrow 9 and thus accordingly in the joining direction 8, in the present case by 0.3 mm, without a deformation of one of the components 22, 52 taking place for this purpose. This mobility counteracts the danger of the connector nozzle 56 becoming jammed when the outlet valve 112 is actuated.

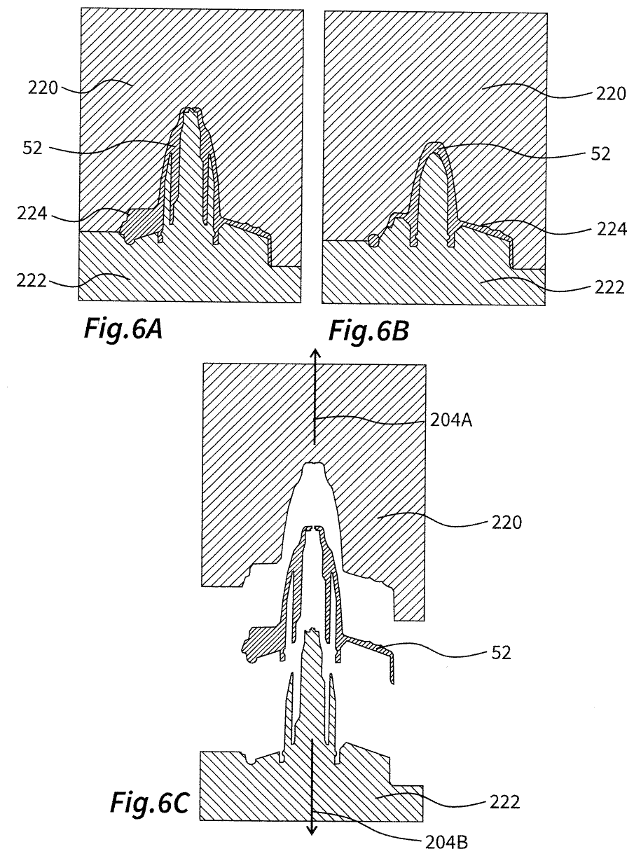

FIGS. 6A to 6C illustrate the injection moulding process for producing the external component 52. The injection moulding process is carried out by means of a single-axis injection mould consisting of two injection moulding segments 220, 222, which together define a cavity 224.

FIGS. 6A and 6B show the injection mould during injection moulding in two different planes. FIG. 6A shows a central sectional plane through the axle carrier 92. FIG. 6B shows an offset sectional plane through one of the axle portions 94. It can be seen that the two injection moulding segments 220, 222 each define the top and the underside of these sub-portions.

FIG. 6C shows the process of separation of the injection moulding segments 220, 222 and thus the demoulding of the external component 52. It can be seen that the injection moulding segments 220, 222 are separated in a uniform separating direction 204A, 204B. Further relative movements of slides or the like are not necessary.

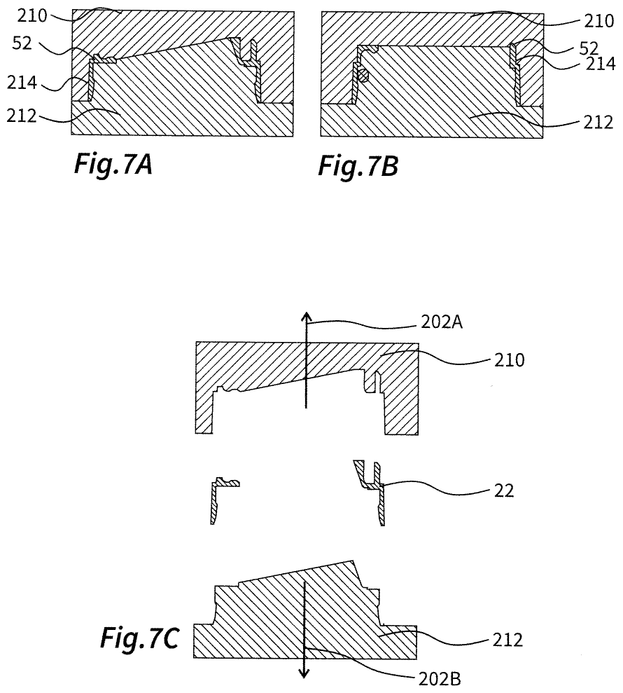

Analogously to FIGS. 6A to 6C, FIGS. 7A to 7C show the production of the base component 22. The injection moulding process is carried out by means of a single-axis injection mould consisting of two injection moulding segments 210, 212, which together define a cavity 214.

FIGS. 7A and 7B show the injection mould during injection moulding in two different planes. FIG. 7A shows a central sectional plane through the central bearing shell portion 36. FIG. 7B shows an offset sectional plane through one of the bearing shell portions 32. It can be seen that the two injection moulding segments 210, 212 each define the top and the underside of these bearing shell portions.

FIG. 7C shows the process of separation of the injection moulding segments 210, 212 and thus the demoulding of the base component 22. It can be seen that, here too, the injection moulding segments 210, 212 are separated in a uniform separating direction 202A, 202B. Further relative movements of slides or the like are not necessary here either.

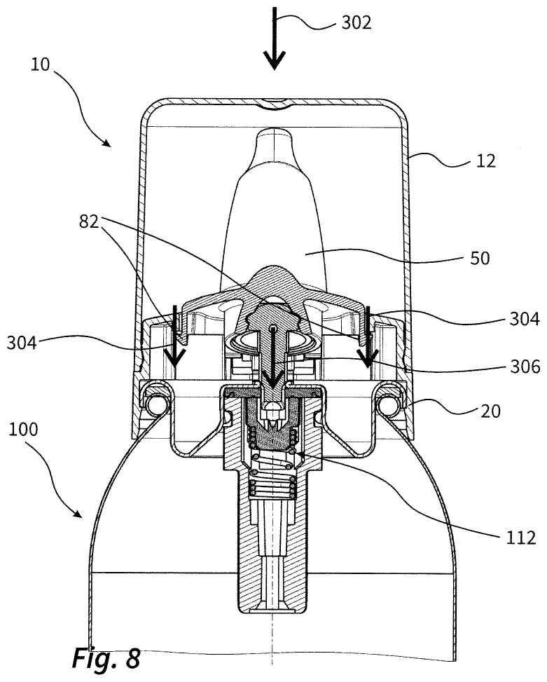

FIG. 8 illustrates a method for mounting the discharge head 10 on a pressure accumulator 110. The discharge head 10, together with a protective cap 12, is pressed from above onto the pressure accumulator 110, the mounting force being introduced in the direction of the arrow 302 into the protective cap 12. This mounting force pushes the coupling device 24 of the base unit 20 over the locking edge 122. At the same time, it acts on the actuating unit 50 via the stop wings 82 and the hinge device 30, 90, as is illustrated by the arrows 304. The mounting force thus acts indirectly, via the stop wings 82, on the connector nozzle 56, which is pressed into the outlet valve 112, as is illustrated by arrow 306.

1. A discharge head for nasal application of liquid from a pressure accumulator, comprising:
a base unit with a coupling device for fastening the discharge head to the pressure accumulator;
a hinge device; and, and
an actuating unit having an elongate nasal applicator extending along an applicator axis, and an actuating surface,
the actuating unit being mounted on the base unit for pivoting movement about a pivot axis by the hinge device such that the actuating unit is pressable downwardly in a pivoting actuating direction to open an outlet valve of the pressure accumulator.

2. The discharge head according to claim 1, wherein
the pivot axis and the actuating surface are arranged on opposite sides of the actuating unit with respect to the nasal applicator.

3. The discharge head according to claim 1, wherein:
the base unit has a base component comprising at least the coupling device and a base-unit-side hinge part device; and
the base component has a component geometry free of non-forcibly demouldable undercuts with respect to a separating direction corresponding to a central axis of the base unit.

4. The discharge head according to claim 1, wherein
the base unit has a base-unit-side hinge part device having at least two outer bearing shell portions and, below the outer bearing shell portions and, with respect to a direction transverse to the applicator axis, between the outer bearing shell portions, an inner bearing shell portion.

5. The discharge head according to claim 1, wherein:
the actuating unit has an external component comprising at least one actuating-unit-side hinge part device and the actuating surface; and
the external component has a component geometry free of non-forcibly demouldable undercuts with respect to a separating direction which corresponds to the applicator axis.

6. The discharge head according to claim 1, wherein
the actuating unit has an actuating-unit-side hinge part device,
the hinge part device has a central axle carrier having a bearing contact surface on an underside thereof, and
the hinge part device has, on both sides of the axle carrier, two axle portions lying opposite each other and having a bearing contact surface on top thereof.

7. The discharge head according to claim 1, wherein
the actuating unit has, on a side facing away from the hinge device, a skirt extending in the direction of the applicator axis.

8. The discharge head according to claim 1, wherein
the actuating unit has at least one outwardly projecting stop wing limiting the mobility of the actuating unit counter to the actuating direction by striking against a corresponding holding surface of the base unit.

9. The discharge head according to claim 1, wherein:
the hinge device has a first hinge part device and a second hinge part device on the base unit and the actuating unit; and
the first hinge part device forms a hinge bearing and has at least two outer bearing shell portions and an inner bearing shell portion arranged opposite the outer bearing shell portions in the direction of the applicator axis, and
the second hinge part device forms a hinge axle and has a central axle carrier mounted on the inner bearing shell portion and two freely projecting axle portions aligned therewith and mounted on the outer bearing shell portions.

10. The discharge head according to claim 1, wherein
the hinge device has a first hinge part device on the base unit and a second hinge part device on the actuating unit, and
a joining direction of the hinge device, in the direction of which the hinge part devices of the actuating unit and of the base unit can be coupled, is angled with respect to a central axis of the base unit.

11. The discharge head according to claim 1, wherein
the discharge head has an actuation protection comprising a protection portion integrally formed on the actuating unit or the base unit, and
the protection portion has at least one destructible sub-portion destroyable by an actuating displacement of the actuating unit with respect to the base unit.

12. The discharge head according to claim 5, wherein:
the actuating unit has an external component forming at least the actuating surface, the actuating-unit-side hinge part device and a sleeve portion of the nasal applicator including a discharge opening; and
the actuating unit has an internal component permanently connected to the external component and forming a line portion directing liquid from the pressure accumulator to the discharge opening.

13. The discharge head according to claim 5, comprising at least one of the following:
the applicator axis is inclined with respect to a central axis of the base unit, wherein, based on an unactuated state, an angle between the central axis and the applicator axis preferably at least 10°; and/or
at least one component of the actuating unit visible in operation and at least one component of the base unit visible in operation have a different colour; and/or
the discharge head has a protective cap which can be latched onto the base unit, and a guide surface on the base unit, in a region of which the protective cap is supported, has in the direction of the central axis an extent of at least 5 mm.

14. A liquid dispenser for discharging liquids, comprising:
a pressure accumulator; a discharge head attached to the pressure accumulator, the discharge head comprising:
a base unit with a coupling device for fastening the discharge head to the pressure accumulator;
a hinge device; and
an actuating unit having an elongate nasal applicator extending along an applicator axis, and an actuating surface, the actuating unit being mounted on the base unit for pivoting movement about a pivot axis by the hinge device such that the actuating unit is pressable downwardly in a pivoting actuating direction to open an outlet valve of the pressure accumulator.

15. A method for producing a base component of a base unit for a discharge head according to claim 1, comprising:
providing the base component with a base-unit-side hinge part device, wherein the hinge part device has at least one lower and at least one upper bearing shell portion;
carrying out the production of the base component by means of an injection moulding process, in which a plurality of injection moulding segments are used and can be displaced along a common opening and separating direction; and
in the course of production, bringing the injection moulding segments together to form a cavity corresponding to the base component, injecting plastic material into the cavity, and, after curing, displacing the injection moulding segments with respect to each other in the common opening and separating direction for the purpose of demoulding.

16. A method for producing an external component of an actuating unit for a discharge head according to claim 1, comprising:
providing the external component with an actuating-unit-side hinge part device, the hinge part device having a central axle carrier with a bearing contact surface on its underside, and the hinge part device has, on both sides of the axle carrier, two axle portions lying opposite each other and having a bearing contact surface on a top thereof;
carrying out production of the external component by means of an injection moulding process using a plurality of injection moulding segments displaceable along a common opening and separating direction; and
in the course of production, bringing the injection moulding segments together to form a cavity corresponding to the external component, injecting plastic material into the cavity, and, after curing, displacing the injection moulding segments with respect to each other in the common opening and separating direction for the purpose of demoulding.

17. A method for mounting a discharge head, according to claim 1, with a base unit and an actuating unit on a pressure accumulator, comprising:
providing the actuating unit with at least one outwardly projecting stop wing and a connector nozzle for transmitting force to an outlet valve of the pressure accumulator, the at least one stop wing limiting the mobility of the actuating unit counter to the actuating direction by striking against a corresponding holding surface of the base unit;
exerting a mounting force on the discharge head, at the base unit, in the direction of the pressure accumulator for the purpose of snapping the discharge head onto a locking edge of the pressure accumulator; and
partially transmitting the mounting force via the at least one stop wing to the actuating unit, in order to ensure coupling of the connector nozzle to the outlet valve of the pressure accumulator.

18. The discharge head according to claim 4, wherein the base unit has a base component on which the base-unit-side hinge part device is disposed, and for demoulding of the base component in injection-moulding production with respect to the central axis, no portions of the base component are present above the inner bearing shell portion and below the outer bearing shell portions.

19. The discharge head according to claim 6, wherein the actuating unit has an external component on which the actuating-unit-side hinge part device is disposed, and for demoulding of the external component in injection-moulding production, with respect to the applicator axis, no portions of the external component are present above the top of the axle portions and below the underside of the axle carrier.

20. The discharge head according to claim 8, further comprising at least one of the following:
the actuating unit has at least two stop wings provided opposite each other on both sides of the actuating unit; and/or
a stop surface of the at least one stop wing is oriented such that a normal vector on the stop surface is inclined with respect to the applicator axis; and/or
the actuating unit has an external component on which the at least one stop wing is disposed, and for demoulding of the external component, with respect to the applicator axis, no portions of the external component are present above and below that of the stop wing.
